(19) United States
(12) Patent Application Publication
(10) Pub. No.: US 2025/0244304 A1
(43) Pub. Date: Jul. 31, 2025

(54) ARTIFICIAL INTELLIGENCE DRIVEN MONITORING SYSTEM FOR AGING WHISKEY

(71) Applicant: BARREL PROOF TECHNOLOGIES LLC, Murfreesboro, TN (US)

(72) Inventor: Brian Richard Anderson, Murfreesboro, TN (US)

(21) Appl. No.: 19/072,871

(22) Filed: Mar. 6, 2025

Related U.S. Application Data
(63) Continuation-in-part of application No. 19/013,859, filed on Jan. 8, 2025.
Application No. 19/013,859 is a continuation-in-part of application No. 18/800,279, filed on Aug. 12, 2024, now Pat. No. 12,228,445.
Application No. 18/800,279 is a continuation-in-part of application No. 18/818,539, filed on Aug. 28, 2024, now Pat. No. 12,228,525.
Application No. 18/800,279 is a continuation-in-part of application No. 18/424,758, filed on Jan. 27, 2024, now Pat. No. 12,117,329.
Application No. 18/818,539 is a continuation-in-part of application No. 18/424,758, filed on Jan. 27, 2024, now Pat. No. 12,117,329.

(51) Int. Cl. G01N 33/14; G01D 9/00; G01D 21/02

(52) U.S. Cl. CPC G01N 33/146; G01D 9/005; G01D 21/02

(57) ABSTRACT

This disclosure relates to an AI-driven, non-invasive system configured to predict whiskey color and maturation outcomes based on sensor-tracked environmental and chemical variables through integrated machine learning, high-accuracy volume sensing, dielectric-based proof monitoring, and environmental data analysis to forecast how whiskey will age inside a barrel over time, and by leveraging real-time barrel data such as time, proof, volume, temperature, humidity, and evaporation rates, the AI model can accurately predict the final color, proof, and optimal aging duration for whiskey and other barrel-aged spirits, which can allow distilleries to reduce inconsistencies, improve yield management, and enhance quality control without relying on manual sampling or invasive testing.

### CROSS-REFERENCE TO RELATED APPLICATION

This application is a Continuation-in-part of, and claims the benefit of U.S. application Ser. No. 19/013,859, titled “SYSTEM AND METHOD FOR DETERMINING FLUID LEVEL AND/OR ALCOHOL CONTENT UTILIZING EXTERNALLY MOUNTED CONTAINER MONITORING SYSTEM” filed on Jan. 8, 2025, which is a Continuation-in part and claims of the benefit of U.S. application Ser. No. 18/800,279, titled “SYSTEM AND METHOD FOR DETERMINING ALCOHOL CONTENT WITHIN CONTAINER UTILIZING CONTAINER MONITORING SYSTEM,” filed on Aug. 12, 2024, and U.S. application Ser. No. 18/818,539, titled “SYSTEM AND METHOD FOR DETERMINING ALCOHOL CONTENT UTILIZING CONTAINER MONITORING SYSTEM,” filed on Aug. 28, 2024, both of which having claimed, as Continuation-in-part applications, the benefit and earlier filing date of U.S. application Ser. No. 18/424,758, titled “CONTAINER MONITORING SYSTEM AND METHOD THEREOF,” filed on Jan. 27, 2024. This application incorporates by reference, herein, the entire contents of the above referred-to patents and applications.

### BACKGROUND OF THE INVENTION

Conventional whiskey aging monitoring systems rely on manual sampling and external measurements to obtain volume, proof, and color assessments of whiskey aging in barrels using periodic sampling and lab testing. However, obtaining whiskey aging data this way results in inaccurate measurements, oxidation risks, and inefficient regulatory compliance because of human error, invasive testing methods, and the lack of real-time insights. Traditional whiskey aging lacks real-time predictive insights-distilleries rely on manual sampling, historical intuition, and periodic lab testing to determine proof, color, and optimal aging time. This results in losses due to over-aging, under-aging, and inconsistencies between barrels.

### SUMMARY OF THE INVENTION

This disclosure relates to an AI-driven, non-invasive system which can be configured to predict whiskey color and maturation outcomes based on sensor-tracked environmental and chemical variables. This disclosure can integrate machine learning, high-accuracy volume sensing, dielectric-based proof monitoring, and environmental data analysis to forecast how whiskey will age inside a barrel over time. By leveraging real-time barrel data such as time, proof, volume, temperature, humidity, and evaporation rates, the AI model can accurately predict the final color, proof, and optimal aging duration for whiskey and other barrel-aged spirits. This can allow distilleries to reduce inconsistencies, improve yield management, and enhance quality control without relying on manual sampling or invasive testing.

These and other advantages of the invention will be further understood and appreciated by those skilled in the art by reference to the following written specification, claims and appended drawings.

### BRIEF DESCRIPTION OF THE DRAWINGS

The advantages, nature, and various additional features of the invention will appear more fully upon consideration of the illustrative embodiments described in detail in connection with the accompanying drawings, where like or similar reference numerals are used to identify like or similar elements throughout the drawings.

FIG. 1 illustrates a first conventional configuration for storing a plurality of barrels and the liquid contained therein.

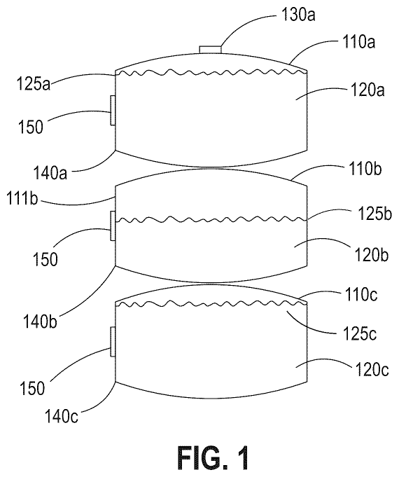

FIG. 2 illustrates a first exemplary embodiment of a system for determining liquid content within a barrel in accordance with the principles of the invention.

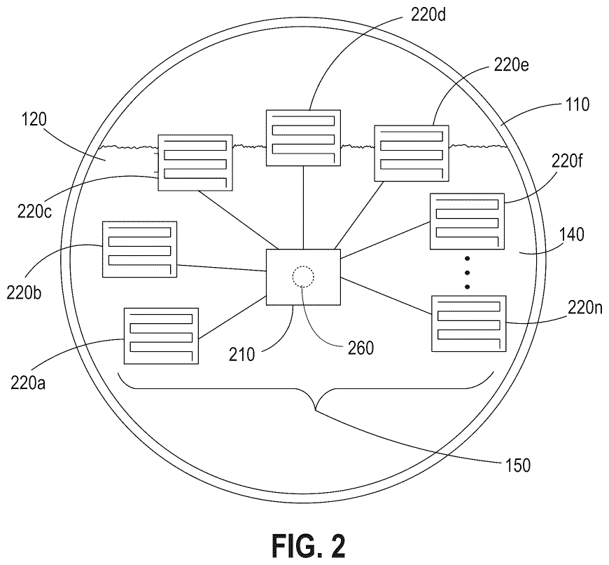

FIG. 3 illustrates a block diagram of an exemplary embodiment of a processing system for determining liquid content within a barrel in accordance with the principles of the invention.

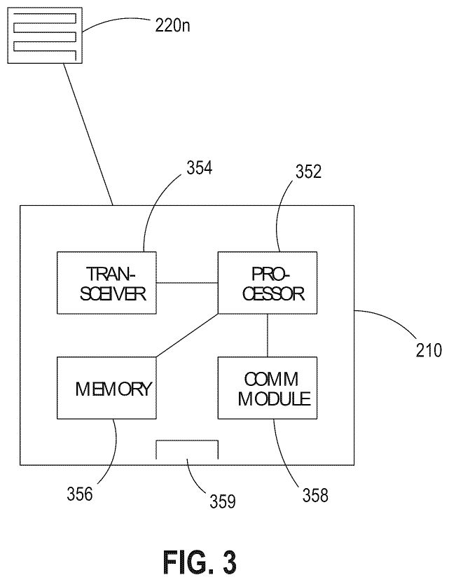

FIG. 4 illustrates a block diagram of an exemplary system for determining liquid content within a barrel in accordance with the principles of the invention.

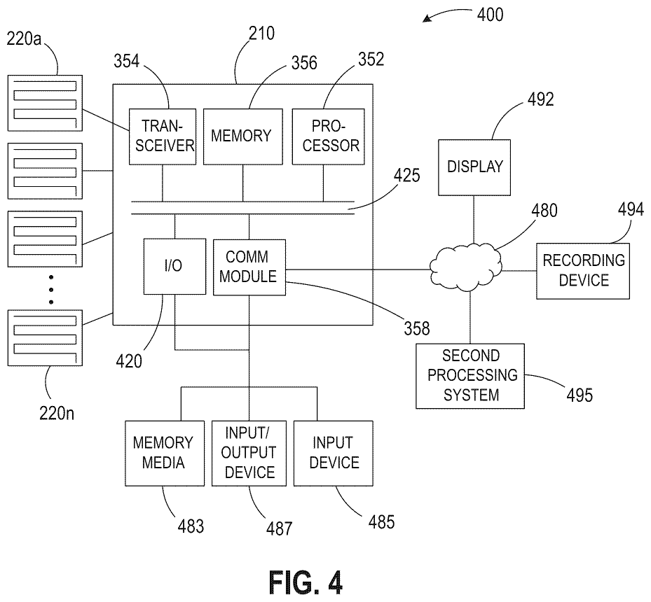

FIG. 5A illustrates a flowchart of an exemplary processing in accordance with the principles of the invention.

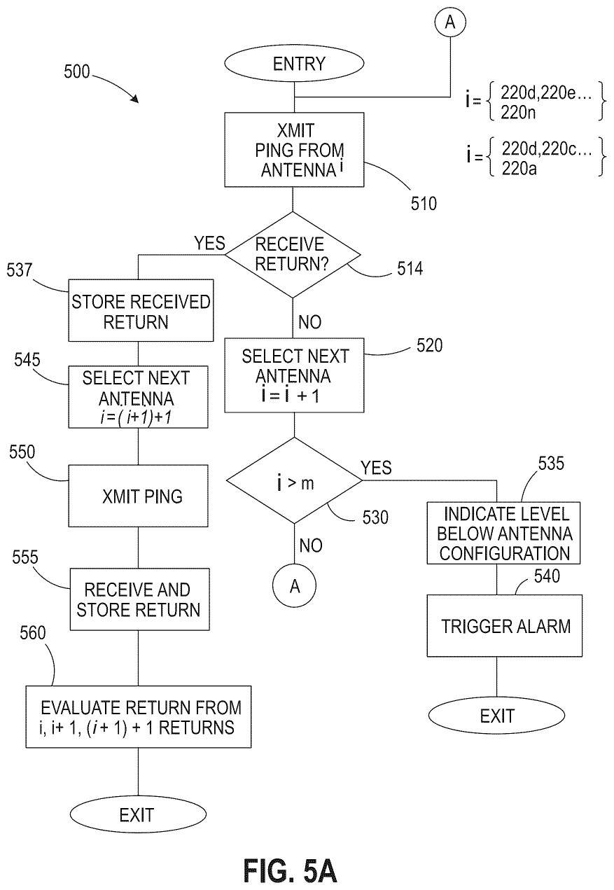

FIG. 5B illustrates an exemplary timing chart in accordance with the principles of the invention.

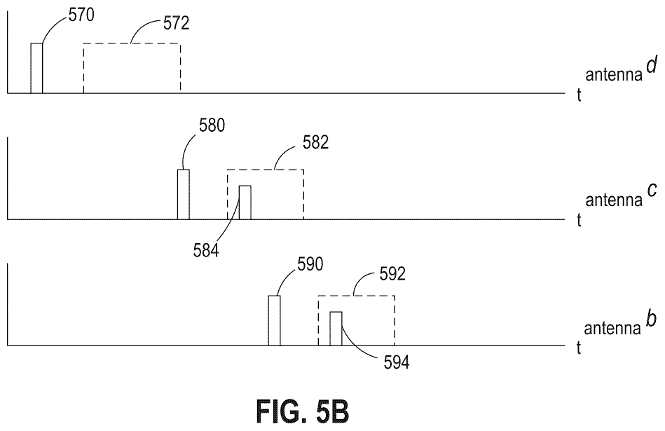

FIG. 6 illustrates a graph of an exemplary signal return chart for determined liquid content within a barrel in accordance with the principles of the invention.

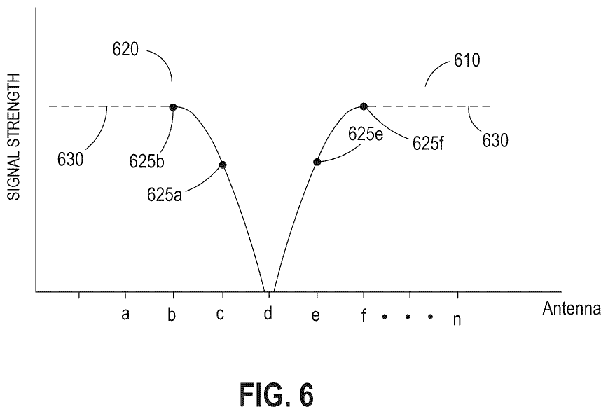

FIGS. 7A and 7B illustrate a first and second aspect of a second exemplary embodiment of a system for determining liquid content within a barrel in accordance with the principles of the invention.

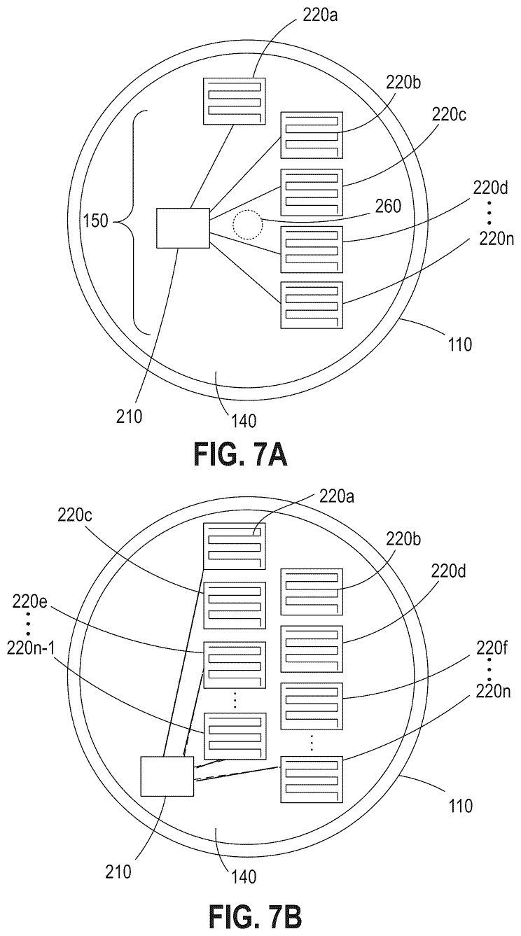

FIG. 8 illustrates a graph of an exemplary signal return chart associated with the configurations shown in FIGS. 7A and 7B for determined liquid content within a barrel in accordance with the principles of the invention.

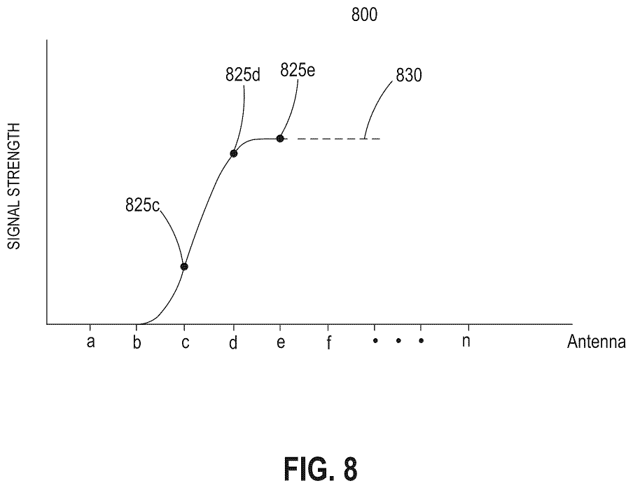

FIG. 9 illustrates a second conventional configuration for storing a plurality of barrels and the liquid contained therein.

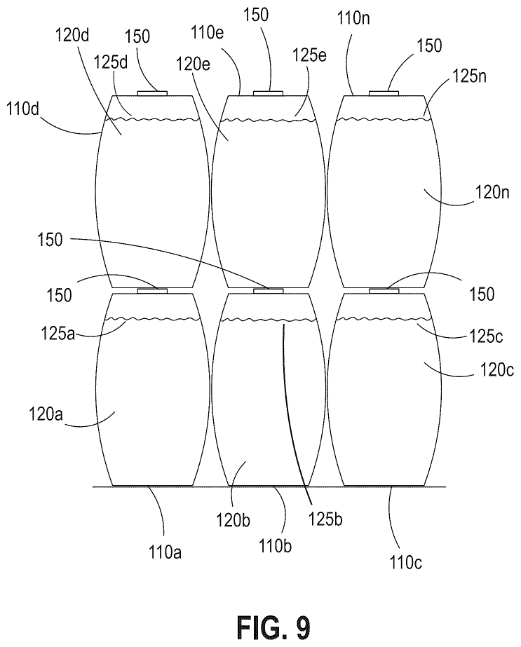

FIG. 10 illustrates a flowchart of an exemplary processing for determining liquid content within a barrel in accordance with the principles of the invention.

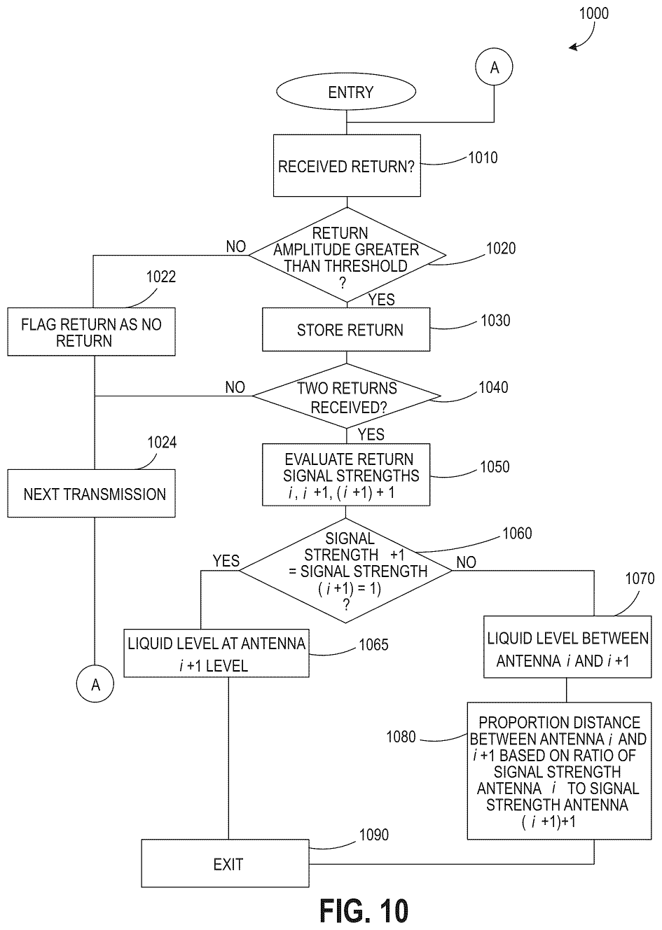

FIGS. 11A-11C illustrate exemplary signal transmission and signal return graphs as a function of time in accordance with one aspect of the invention.

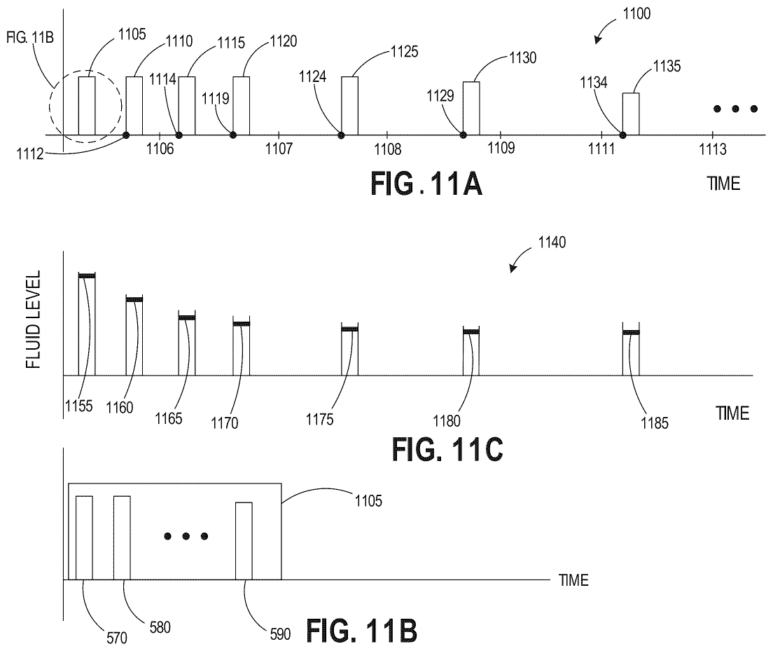

FIG. 12 illustrates an exemplary processing associated with the graphs shown in FIGS. 11A and 11B.

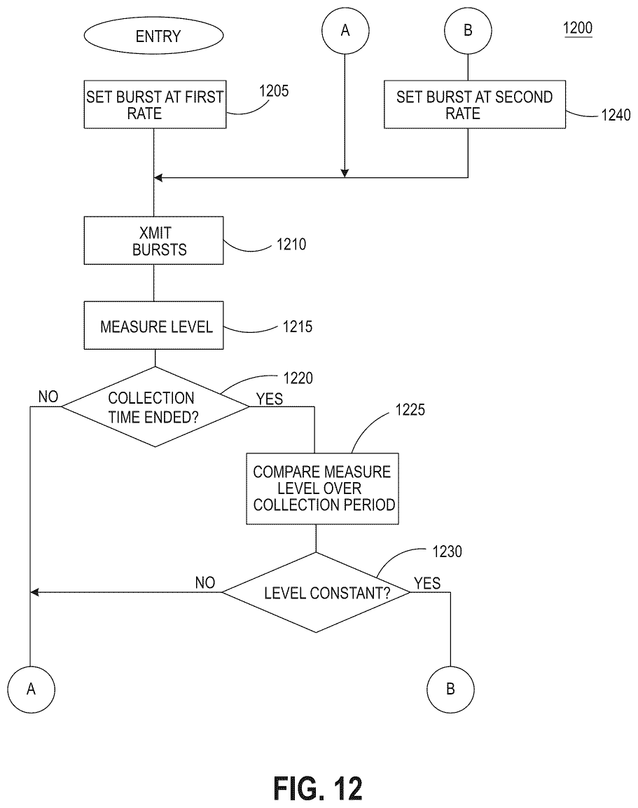

FIG. 13 illustrates a flowchart of an exemplary process associated with a determination of an alcohol content within a container in accordance with the principles of the invention.

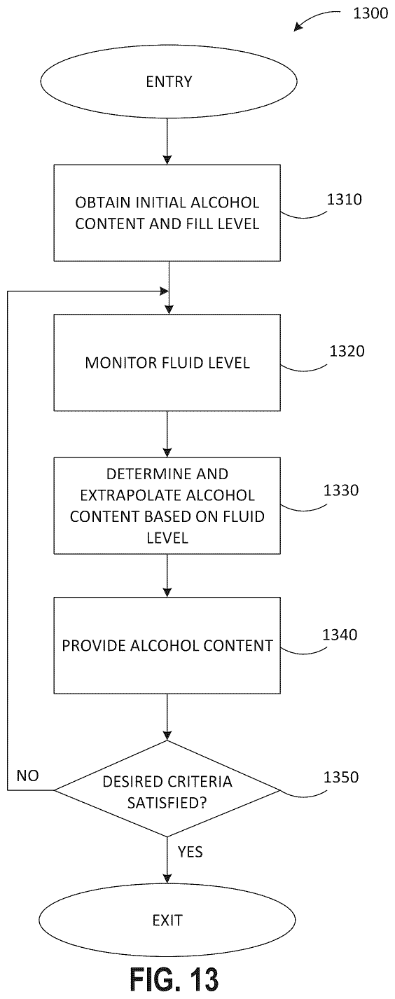

FIG. 14 illustrates a flowchart of an exemplary process for determining and extrapolating alcohol content of a liquid within a container in accordance with the principles of the invention.

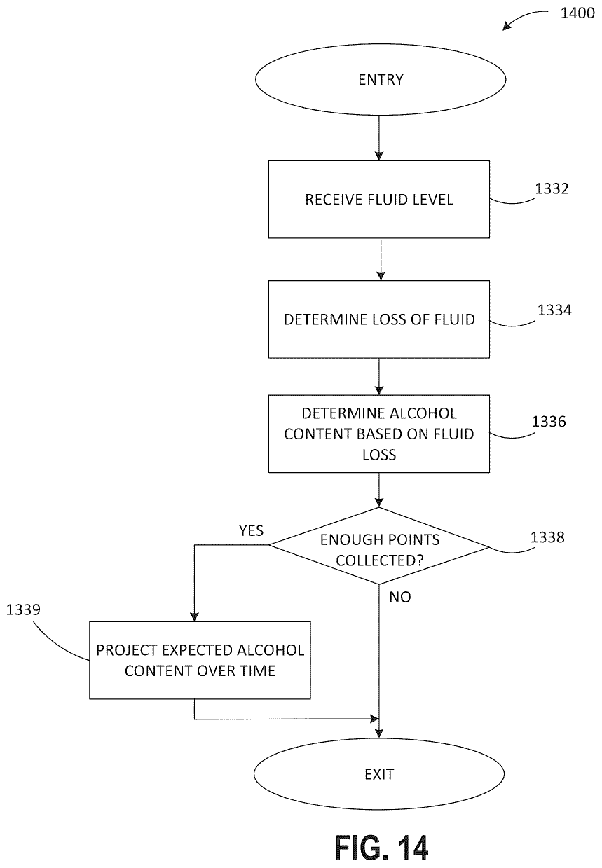

FIG. 15 illustrates a flowchart of an exemplary process for determining alcohol content of a liquid within a container in accordance with the principles of the invention.

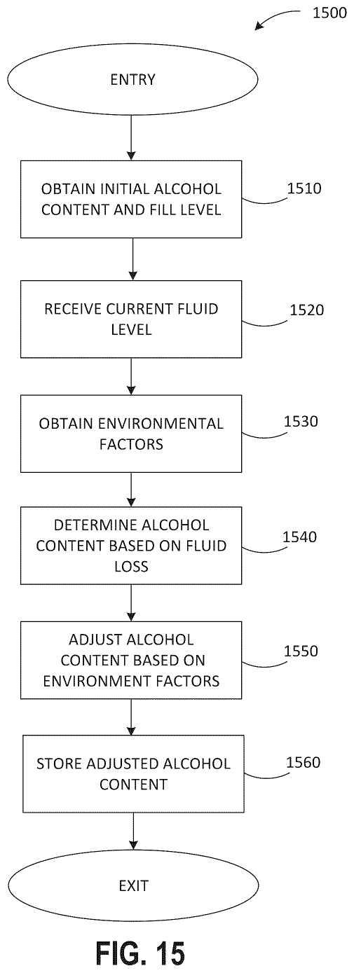

FIG. 16 illustrates a flowchart of an exemplary process for adjusting a determined alcohol content based on environmental considerations.

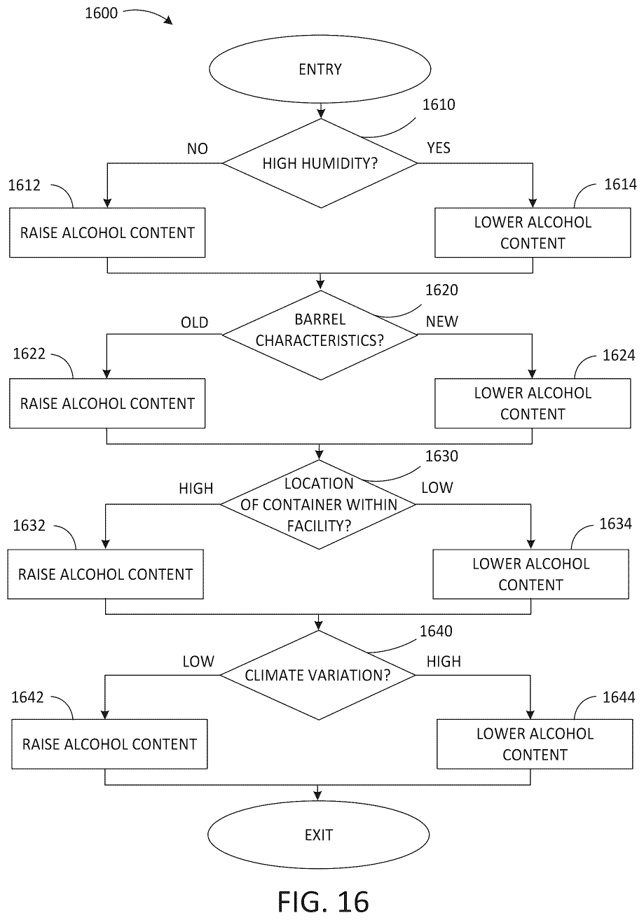

FIG. 17 illustrates an exemplary plotting and extrapolating alcohol content of a liquid within a container in accordance with the principles of the invention.

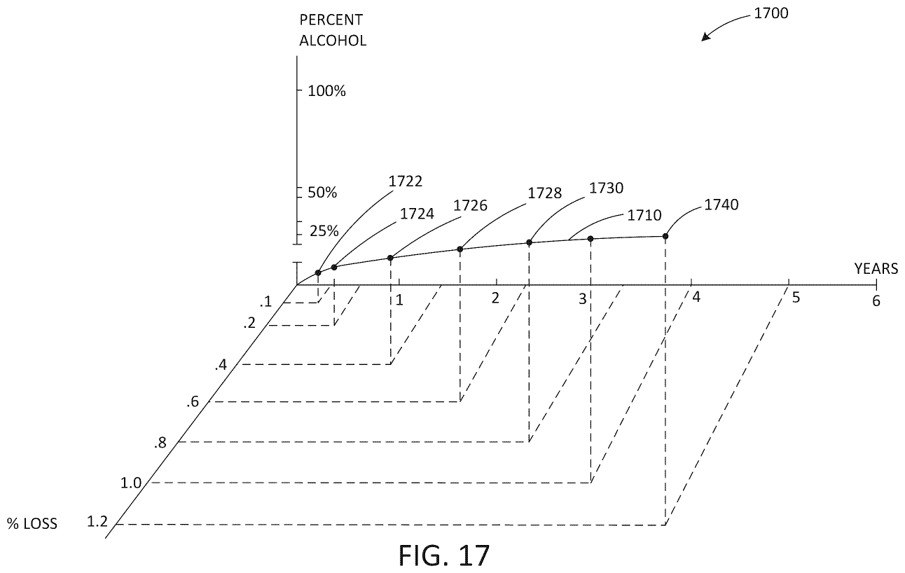

FIG. 18 illustrates an exemplary graph of alcohol content determination in accordance with the principles of the invention.

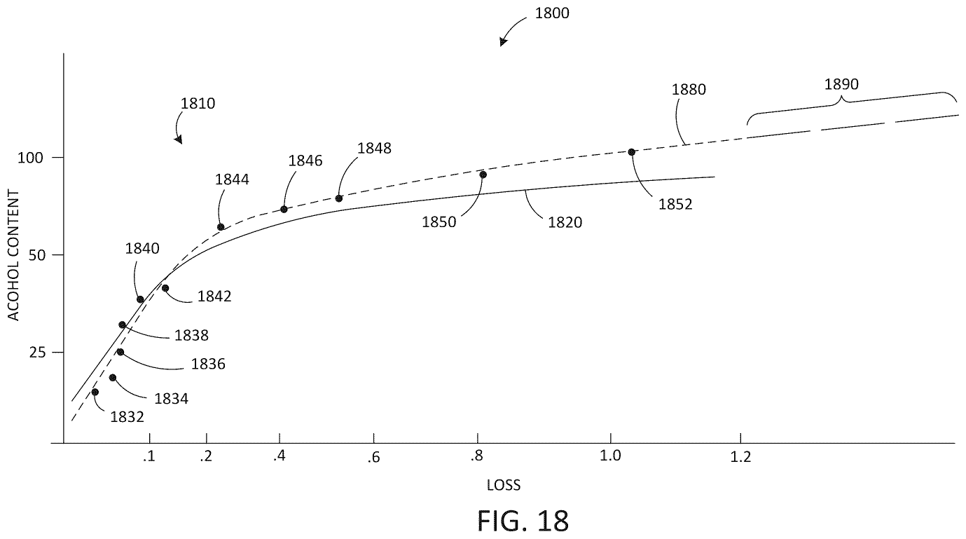

FIG. 19 illustrates a flowchart of a second exemplary process associated with a determination of an alcohol content within a container in accordance with the principles of the invention.

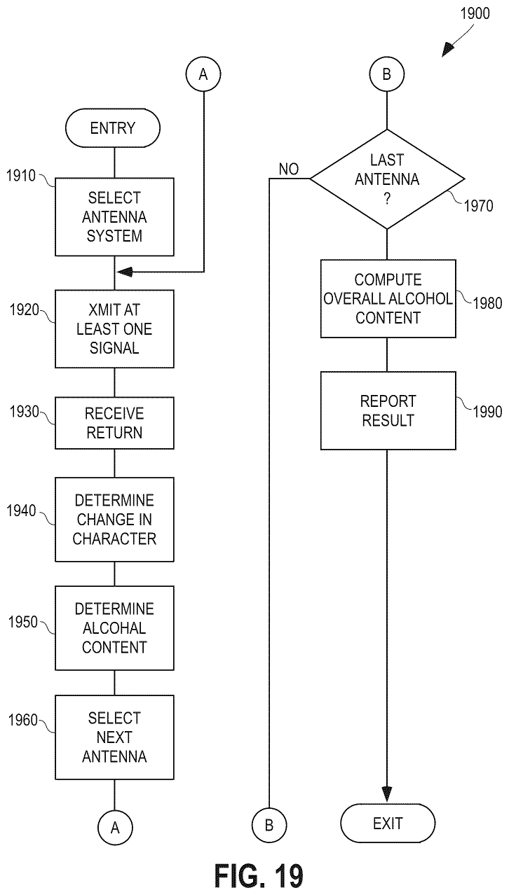

FIGS. 20A-D illustrate exemplary charts of alcohol content as a function of change in frequency.

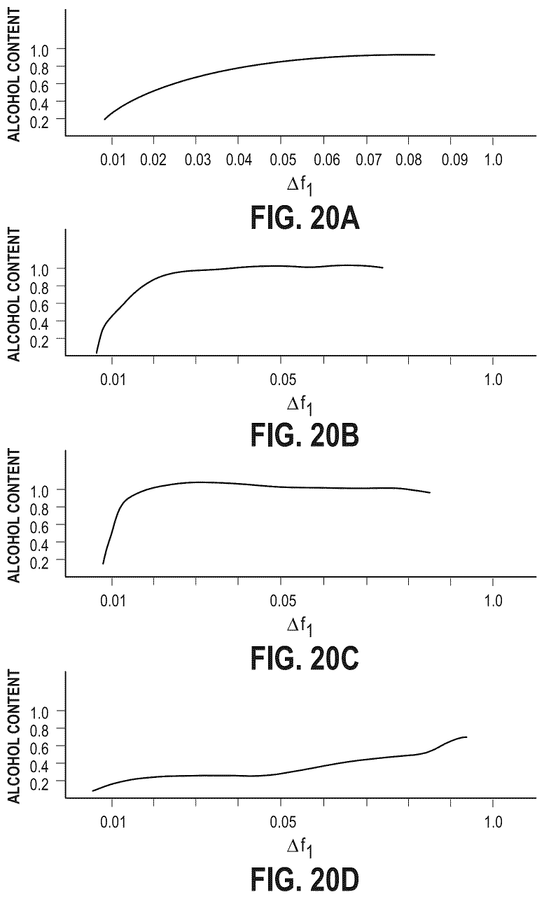

FIG. 21 illustrates an exemplary chart of measurement of alcohol content as a function of time.

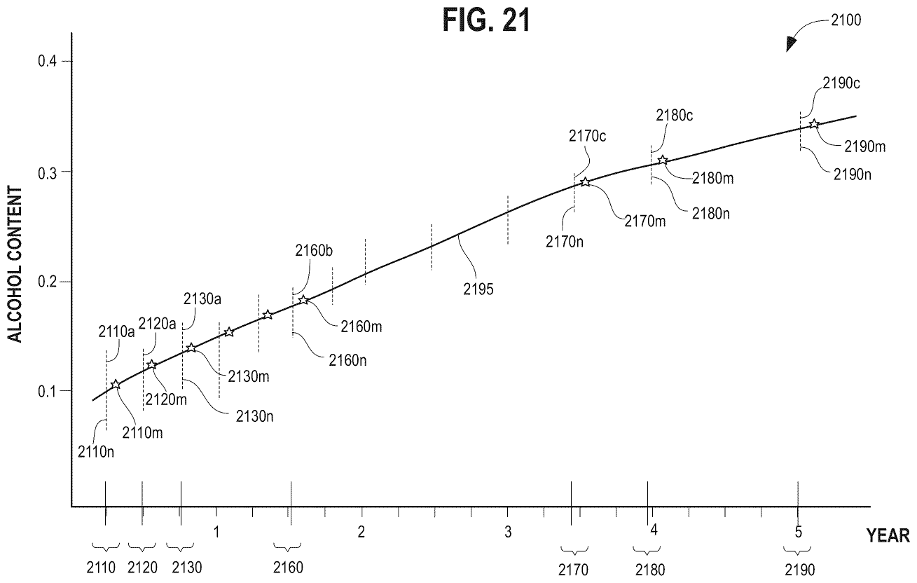

It is to be understood that the figures, which are not drawn to scale, and descriptions of the present invention described herein have been simplified to illustrate the elements that are relevant for a clear understanding of the present invention, while eliminating, for purposes of clarity, many other elements. However, because these omitted elements are well-known in the art, and because they do not facilitate a better understanding of the present invention, a discussion of such elements are not provided herein. The disclosure, herein, is directed also to variations and modifications known to those skilled in the art.

### DETAILED DESCRIPTION OF THE PREFERRED EMBODIMENT(S)

The artificial intelligence driven whiskey prediction system can use an artificial intelligence (hereafter referred to as “AI”) powered, non-invasive barrel monitoring system to prevent inefficiency by using radar-based volume tracking, dielectric proof sensing, and machine-learning color prediction to provide real-time whiskey aging insights without opening barrels. In some embodiments, the aging predictions and compliance measurements obtained by the AI-driven system can be more accurate, automated, and scalable, as a result of our integrated AI, radar, and sensor technology design that analyzes environmental conditions, evaporation rates, and proof fluctuations continuously. The AI-driven whiskey monitoring system can be used in distilleries, spirits manufacturing, and regulatory agencies for producing higher-quality, consistent, and efficiently taxed whiskey while reducing waste and optimizing aging strategies.

The AI-driven whiskey prediction system can integrate radar-based liquid volume tracking, dielectric proof sensing, and AI-driven predictive analytics into a non-invasive, real-time system that eliminates the need for manual sampling. The system can be configured to detect barrel aging conditions through sensor inputs. These inputs can comprise of radar-based frequency-modulated continuous wave (FMCW) signals which can penetrate the wooden barrel to measure liquid volume changes accurately over time. These inputs can also include a dielectric-based sensor array configured to detect fluctuations in ethanol concentration (proof) by analyzing electromagnetic permittivity within the barrel substrate. These inputs can include temperature and humidity sensors configured to collect environmental data affecting whiskey aging.

The system can be configured to include processing the detected data using an AI-driven model. The processed data can include volume, proof, temperature, and humidity data with an AI-trained predictive aging model. The processing can include using a machine learning (hereinafter referred to as “ML”) model, such as convolutional neural networks (hereinafter referred to as “CNN”) and long short-term memory (hereinafter referred to as “LSTM”), analyzes historical and real-time barrel data to forecast whiskey color evolution over time. The processing can use AI to correlate whiskey evaporation trends with proof adjustments to ensure compliance with regulatory taxation standards.

The system can be configured to include triggering automated aging optimization and compliance actions. The system can be configured to generate a digital twin of barrel aging conditions, enabling distilleries to make real-time, AI-driven decisions on aging, blending, and bottling. The actions can include a compliance automation module generates tax-ready reports that match regulatory requirements (e.g., TTB standards). In some embodiments, the system can alert operators if a barrel has reached optimal maturation based on AI-driven predictions, preventing over-aging or premature bottling.

In some embodiments, the CNN-LTSM hybrid system can use time-series aging trends and spectrophotometric features to forecast color evolution non-invasively. In some embodiments, the machine learning system can use reinforcement learning to do proof calibration. The proof calibration can adapt dielectric permittivity models to environmental fluctuations to ensure real-time ethanol concentration tracking. In some embodiments, the machine learning system can use gaussian process regression for barrel evaporation. The gaussian process regression can compensate for warehouse-specific evaporation rates and can improve proof measurement accuracy. In some embodiments, the machine learning system can use autoencoder-based anomaly detection for tax compliance to detect fraud risks and underreported whiskey losses.

In some embodiments, the AI-driven whiskey prediction system can include a ML model which can calibrate dielectric ethanol concentration tracking based on changes in temperature and humidity. In some embodiments, the gaussian process regression (hereinafter referred to as “GPR”) can be configured to model nonlinear correlations between whiskey proof fluctuations and barrel evaporation rates. In some embodiments, the adaptive ML can be configured to compensate for barrel variations (wood grain, char level, warehouse location).

In some embodiments, the AI-driven whiskey prediction system can include a monitoring system configured to include a radar-based volume detection module. The radar-based volume detection module can be configured to emit frequency-modulated continuous wave (FMCW) radar signals into a sealed wooden barrel. The module can also be configured to receive signal reflections and process liquid volume changes inside the barrel without physical contact. The module can also be configured to generate real-time volumetric loss data indicative of evaporation trends (i.e. Angel's Share). In some embodiments, the radar-based volume detection module can apply adaptive signal filtering algorithms to compensate for interference caused by barrel char thickness and wood grain variability.

In some embodiments, the AI-driven whiskey prediction system can include a dielectric-based proof monitoring module. The monitoring module can be configured to detect ethanol concentration (proof) within the barrel using dielectric permittivity measurements of the substrate. The monitoring module can also be configured to calculate real-time fluctuations in proof due to evaporation without requiring sample extraction. The monitoring module can also be configured to generate continuous proof tracking data to ensure compliance with regulatory standards. In some embodiments, the dielectric-based proof monitoring module can dynamically adjust ethanol concentration calculations based on real-time temperature and humidity variations.

In some embodiments, the AI-driven whiskey prediction system can include a convolutional neural network (CNN) and long short-term memory (LSTM) model trained on historical whiskey aging data. The CNN and LSTM models can include an input interface to receive time-series data from radar, dielectric, temperature, and humidity sensors. The CNN and LSTM models can also include a processing engine configured to predict future whiskey color evolution and optimal maturation timelines based on proof, volume, and environmental conditions.

In some embodiments, the AI-driven whiskey prediction system can include a compliance automation module. The compliance automation module can be configured to generate automated tax compliance reports in accordance with Alcohol and Tobacco Tax and Trade Bureau (TTB) standards. The compliance automation module can also be configured to log real-time proof, volume, and aging adjustments in a secure digital ledger. The compliance automation module can also be configured to provide regulatory audit-ready reports to distilleries and government agencies. In some embodiments, the compliance automation module can implement a blockchain-based verification system for secure storage of tax compliance data. In some embodiments, the compliance automation module can implement a real-time anomaly detection algorithm to identify potential discrepancies in reported proof and volume data. In some embodiments, the compliance automation module can implement an auto-generated regulatory filing system that directly submits compliance reports to government agencies.

In some embodiments, the AI-driven whiskey prediction system can include a remote monitoring interface. The remote monitoring interface can include a cloud-based platform for distillery operators to visualize real-time whiskey aging conditions. The remote monitoring interface can include an alert system that triggers notifications when a barrel has reached optimal aging, proof changes beyond a threshold, or unexpected evaporation occurs. The remote monitoring interface can also include an API for integrating with third-party distillery management systems. In some embodiments, the remote monitoring interface can integrate with IoT-enabled warehouse sensors to optimize barrel placement. In some embodiments, the remote monitoring interface can provide real-time analytics dashboards for monitoring multiple aging warehouses. In some embodiments, the remote monitoring interface can allow distillery operators to remotely adjust storage conditions based on AI-driven recommendations.

In some embodiments, the AI-driven whiskey prediction system can include an AI-driven whiskey aging prediction module. The whiskey aging prediction module can be trained on historical distillery datasets of color change trends, configured to use machine learning feature selection to identify correlations between aging conditions and whiskey hue evolution, and configured to predict a future color profile based on proof, volume, and storage conditions.

In some embodiments, the AI-driven whiskey prediction system can include a computer-implemented method for monitoring and optimizing whiskey aging. The method can include receiving real-time sensor data, including radar-based volume readings, dielectric based proof measurements, and environmental conditions. The method can also include processing the received data using an AI-driven predictive model. The processing can be configured to determine current whiskey aging parameters, predict future color evolution based on time-series data, and optimize barrel storage conditions for yield maximization. In some embodiments, the method can include generating automated compliance reports. The reports can include volume loss adjustments for tax filing, proof fluctuation records, and regulatory audit-ready logs for government agencies. In some embodiments, the method can include triggering actionable insights. The actionable insights can include alerts for optimal aging readiness, notifications for unexpected evaporation losses, and recommendations for blending and bottling decisions.

In some embodiments, the AI-driven whiskey prediction system can use an ultrasonic transducer to measure liquid volume inside the barrel by detecting sound wave reflections. In some embodiments, the AI-driven whiskey prediction system can include near-infrared (hereinafter “NIR”) sensors to analyze alcohol content. In some embodiments, the AI-driven whiskey prediction system can allow for color to be measured via extracted samples using UV-Vis spectroscopy. In some embodiments, the AI-driven whiskey prediction system can include systems to simply log temperature and humidity data without predictive modeling to predict optimal aging durations. In some embodiments, the AI-driven whiskey prediction system can include users spreadsheets and regulatory filing software.

In some embodiments, the AI-driven whiskey prediction system can use dielectric property changes to track ethanol concentration (proof) inside the barrel without invasive sampling. In some embodiments, ultrasonic sensing can be used for proof estimation. In some embodiments, the ultrasonic technologies can detect liquid density changes, which could be correlated to proof levels. In some embodiments, an external optical system can be used to track changes in the refractive index of whiskey, which correlates with ethanol content, but this would require light penetration into the barrel. In some embodiments, vapor phase ethanol sensors can be used to estimate proof based on the concentration of ethanol in the barrel's headspace.

In some embodiments, ultrasonic level sensors can be used measure liquid height in the barrel. In some embodiments, pressure sensors can be used to estimate volume by tracking pressure changes at the barrel base, but this would require physical modifications to barrels. In some embodiments, capacitive sensors can be used to detect liquid presence by measuring electrical capacitance, but would be less precise than radar in wooden barrel environments.

In some embodiments, the AI-driven whiskey prediction system can use IoT-enabled temperature & humidity sensors to track warehouse conditions affecting aging rates. In some embodiments, infrared temperature sensors can be used to monitor warehouse temperature distribution. In some embodiments, the AI-driven whiskey prediction system can use direct control over temperature & humidity through automated HVAC systems could optimize aging.

In some embodiments, the AI-driven whiskey prediction system can provide real-time monitoring & automated alerts when a barrel reaches optimal aging conditions. In some embodiments, the AI-driven whiskey prediction system can be configured to generate weekly or monthly reports, but this would delay optimization decisions. In some embodiments, the AI-driven whiskey prediction system can be configured to calculate the best aging duration based on sensor inputs to maximize whiskey quality and yield. In some embodiments, the AI-driven whiskey prediction system can use a predefined set of aging rules. In some embodiments, the AI-driven whiskey prediction system can be configured to allow distillers to input desired outcomes manually rather than relying on Al learning.

In some embodiments, the AI-driven whiskey prediction system can include a method for monitoring and tracking a distilling liquid. The method can include monitoring a distilling liquid with at least one sensor. The method can include collecting at least one distilling datapoint from the at least one sensor. The method can include receiving and analyzing the at least one distilling datapoint via an artificial intelligence model. The method can include determining via the artificial intelligence model at least one distilling characteristic of the distilling liquid. The method can include transmitting the at least one distilling characteristic to a user.

In some embodiments, the distilling datapoint can be at least one of a volume, a color, a proof, a temperature, a humidity, and an evaporation rate. In some embodiments, the at least one sensor can be at least one of a radar sensor, a dielectric sensor, an ultrasonic transducer, an environmental sensor, a near infrared sensor, and an ultraviolet-visible sensor. In some embodiments, the method can include tracking the at least one distilling datapoint and the at least one distilling characteristic. In some embodiments, the method can include comparing the at least one distilling datapoint and the at least one distilling characteristic, via the artificial intelligence model, against a user-input dataset. In some embodiments, the method can include calculating an optimal aging duration and a bottling time via the artificial intelligence model.

In some embodiments, the method can include alerting the user that the distilling liquid is in an ideal aging window, wherein the ideal aging window is in the user-input dataset. In some embodiments, the method can include determining a distilling recommendation for an optimal aging time and a yield improvement via the artificial intelligence model. In some embodiments, the method can include transmitting the distilling recommendation to the user. In some embodiments, the distilling liquid is contained within a barrel. In some embodiments, the at least one sensor can be a non-invasive sensor. In some embodiments, the method can include tracking at least one of the volume, the proof, and an evaporation loss of the distilling liquid. In some embodiments, the method can include generating a compliance report via the artificial intelligence model. In some embodiments, the method can include transmitting the compliance report to the user.

In some embodiments, the AI-driven whiskey prediction system can include an apparatus for monitoring and tracking a distilling liquid. The apparatus can include at least one sensor configured to measure at least one distilling datapoint of a distilling liquid. The apparatus can include a computing device configured to receive the at least one distilling datapoint from the at least one sensor and analyze the at least one distilling datapoint via an artificial intelligence model. In some embodiments, the artificial intelligence model can be configured to determine at least one distilling characteristic of the distilling liquid. In some embodiments, the artificial intelligence model can be configured to track the at least one distilling datapoint and at least one distilling characteristic.

It is to be understood that the specific devices and processes illustrated in the attached drawings and described in the following specification are exemplary embodiments of the inventive concepts defined in the appended claims. Hence, specific dimensions and other physical characteristics relating to the embodiments disclosed herein are not to be considered as limiting, unless the claims expressly state otherwise.

In the foregoing description, it will be readily appreciated by those skilled in the art that modifications may be made to the invention without departing from the concepts disclosed herein. Such modifications are to be considered as included in the following claims, unless the claims by their language expressly state otherwise.

Variations described for exemplary embodiments of the present invention can be realized in any combination desirable for each particular application. Thus, particular limitations, and/or embodiment enhancements described herein, which may have particular limitations, need be implemented in methods, systems, and/or apparatuses including one or more concepts describe with relation to exemplary embodiments of the present invention.

Therefore, it is intended that the invention not be limited to the particular embodiments disclosed as the best mode contemplated for carrying out this invention, but that the invention will include all embodiments falling within the scope of the present application as set forth in the following claims, wherein reference to an element in the singular, such as by use of the article “a” or “an” is not intended to mean “one and only one” unless specifically so stated, but rather “one or more.” Moreover, no claim element is to be construed under the provisions of 35 U.S.C. § 112, sixth paragraph, unless the element is expressly recited using the phrase “means for” or “step for.” These following claims should be construed to maintain the proper protection for the present invention.

In the foregoing description, it will be readily appreciated by those skilled in the art that modifications may be made to the invention without departing from the concepts disclosed herein. Such modifications are to be considered as included in the following claims, unless the claims by their language expressly state otherwise.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future.

Likewise, where this document refers to technologies that would be apparent or known to one of ordinary skill in the art, such technologies encompass those apparent or known to the skilled artisan now or at any time in the future. Furthermore, the use of plurals can also refer to the singular, including without limitation when a term refers to one or more of a particular item; likewise, the use of a singular term can also include the plural, unless the context dictates otherwise.

The presence of broadening words and phrases such as “one or more,” “at least,” “but not limited to” or other like phrases in some instances shall not be read to mean that the narrower case is intended or required in instances where such broadening phrases may be absent. Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

There has thus been outlined, rather broadly, the more important features of the disclosure in order that the detailed description thereof may be better understood, and in order that the present contribution to the art may be better appreciated. There are additional features of the disclosure that will be described hereinafter, and which will also form the subject matter of the claims appended hereto. The features listed herein, and other features, aspects and advantages of the present disclosure will become better understood with reference to the following description and appended claims.

While various embodiments of the present disclosure have been described above, it should be understood that they have been presented by way of example only, and not of limitation. Likewise, the various diagrams may depict an example architectural or other configuration for the invention, which is provided to aid in understanding the features and functionality that can be included in the invention. The invention is not restricted to the illustrated example architectures or configurations, but the desired features can be implemented using a variety of alternative architectures and configurations.

Indeed, it will be apparent to one of skill in the art how alternative functional configurations can be implemented to implement the desired features of the present disclosure. Additionally, with regard to flow diagrams, operational descriptions and method claims, the order in which the steps are presented herein shall not mandate that various embodiments be implemented to perform the recited functionality in the same order unless the context dictates otherwise.

Although the disclosure is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present invention should not be limited by any of the above-described exemplary embodiments.

What is claimed is:

1. A method comprising:
monitoring a distilling liquid with at least one sensor;
collecting at least one distilling datapoint from the at least one sensor;
receiving and analyzing the at least one distilling datapoint via an artificial intelligence model;
determining via the artificial intelligence model at least one distilling characteristic of the distilling liquid; and
transmitting the at least one distilling characteristic to a user.

2. The method of claim 1, wherein the distilling datapoint is at least one of a volume, a color, a proof, a temperature, a humidity, and an evaporation rate.

3. The method of claim 1, wherein the at least one sensor is at least one of a radar sensor, a dielectric sensor, an ultrasonic transducer, an environmental sensor, a near infrared sensor, and an ultraviolet-visible sensor.

4. The method of claim 1, further comprising:
tracking the at least one distilling datapoint and the at least one distilling characteristic; and
comparing them, via the artificial intelligence model, against a user-input dataset.

5. The method of claim 4, further comprising, calculating an optimal aging duration and a bottling time via the artificial intelligence model.

6. The method of claim 5, further comprising alerting the user that the distilling liquid is in an ideal aging window, wherein the ideal aging window is in the user-input dataset.

7. The method of claim 6, further comprising:
determining a distilling recommendation for an optimal aging time and a yield improvement via the artificial intelligence model; and
transmitting the distilling recommendation to the user.

8. The method of claim 1, wherein:
the distilling liquid is contained within a barrel; and
the at least one sensor is non-invasive.

9. The method of claim 2, further comprising:
tracking at least one of the volume, the proof, and an evaporation loss of the distilling liquid;
generating a compliance report via the artificial intelligence model; and
transmitting the compliance report to the user.

10. An apparatus, comprising:
at least one sensor configured to measure at least one distilling datapoint of a distilling liquid; and
a computing device configured to receive the at least one distilling datapoint from the at least one sensor and analyze the at least one distilling datapoint via an artificial intelligence model, wherein:
the artificial intelligence model is configured to determine at least one distilling characteristic of the distilling liquid; and
the artificial intelligence model is configured to track the at least one distilling datapoint and at least one distilling characteristic.
